(19) United States
(12) Patent Application Publication
(10) Pub. No.: US 2002/0187995 A1
(43) Pub. Date: Dec. 12, 2002

(54) Methods for use in modifying the perception of pain

(76) Inventors: Desmond Joseph Fitzgerald, Howth (IE); Connail Rogers McCrory, Dublin (IE)

Correspondence Address: BIRCH STEWART KOLASCH & BIRCH, PO BOX 747, FALLS CHURCH, VA, 22040-0747, US

(21) Appl. No.: 09/845,277

(22) Filed: May 1, 2001

(51) Int. Cl. A61K 31/485

(52) U.S. Cl. 514/282

(57) ABSTRACT

Use of a substance which is COX-2 selective and which can reduce prostaglandin levels of cerebrospinal fluid in a method for reducing the perception of pain in a subject is described. A preferred COX-2 selective inhibitor is nimesulide. Use of a COX-2 selective inhibitor as described has particular application in the relief of pain following surgery, especially following thoractotomy and also in the treatment of chronic post-thoracotomy pain.

### TECHNICAL FIELD

This invention relates to medicaments for use in the treatment of pain and, in particular, the perception of pain by nociceptors.

### BACKGROUND ART

Prostaglandins regulate a number of neurological functions, and in particular may have a role in spinal nociceptive processing. Increased spinal generation of prostaglandins has been reported following afferent nerve stimulation or peripheral injury (Zhao, Z. et al Neuroscience (2000); 97:743-8). Moreover, intrathecal administration of COX inhibitors reduces the hyperalgesic response that follows thermal injury. Prostacyclin may play an important role in this respect. Prostacyclin (IP) receptors are expressed in high density at nerve terminals in spinal neurones where they may enhance neurotransmission (Bley, K. R. et al Trends Pharmacol. Sci. (1998); 19:141-7). Moreover, disruption of the IP receptor in mice attenuates the writhing response to peripheral tissue injury (Murata, T. et al Nature (1997); 388:678-82).

There is strong experimental evidence that prostaglandins acting within the spinal cord or at higher levels facilitate pain perception. The prostaglandins (PGs), PGE₂ and prostacyclin, are generated in the central nervous system in response to peripheral injury. PGs enhance nociceptive neurotransmission and their local application in the spine induces pain responses (Evans, A. R. et al J. Pharmacol. Exp. Ther. (2000); 293:912-20). Each of the enzymes required for PG synthesis and receptors for PGE₂ and prostacyclin are found in dorsal root ganglia (Bley, K. R. et al supra).

PGs are generated from arachidonic acid by the enzyme cyclooxygenase (COX), of which there are two isoforms. COX-1 is present in most cells while COX-2 is expressed to only a limited extent in normal tissues, including the brain.

The facilitation of the processing of nociceptive signals by PGs in the spine may explain the analgesic effects of non-steroidal antiinflammatory drugs (NSAIDs) following surgery. NSAIDs are known to reduce the amount of opioids required for effective analgesia.

Both COX isoforms are found in the normal brain and spinal cord (Kaufman, W. E. et al Proc. Natl Acad Sci USA (1996); 93:2317-21), but COX-2 may be the principal isoform responsible for the generation of prostaglandins following peripheral injury. Enhanced COX-2 expression has been reported in the spinal cord following adjuvant-induced arthritis or other forms of injury in the rat (Ebersberger, A. Neuroscience (1999); 93(2):775-81). Several studies have also reported an analgesic response to the intrathecal administration of a COX-2 inhibitor in experimental models (Yamamoto, T. and Nozaki-Taguchi, N. Neuroreport (1997); 8:2179-82). Thus, spinal COX-2 expression may be enhanced following a peripheral injury and contribute to the perception of pain. However, pain responses were attenuated in mice lacking the COX-1 isoform, whereas disruption of COX-2 had no effect (Ballou, L. R. Proc Natl Acad Sci USA (2000); 97:10272-10276). Consequently, it is unclear which isoform is responsible for the clinical response to NSAIDs in man.

Nimesulide is a selective COX-2 inhibitor (Shah, A. A. et al Rheumatology (1999); 38 Suppl 1: 19-23) and thus acts as a cyclooxygenase inhibitor.

Post-thoracotomy pain is one of the most intense pain experiences known being composed of both nociceptive and neuropathic components that are relatively insensitive to opioid analgesia (Arner, S. and Meyerson, B. Pain Dig (1993); 3:15-22). At one year following surgery 61% of patients will complain of chronic post-thoracotomy pain (Pertufunen, K. et al Acta Anaesthesiol Scand. (1999); 43:563-7). Longer follow-up studies report 30% of patients complaining of persistent chest wall pain at four years (Dajczman, E. et al Chest (1991); 99:270-4). Inadequately treated post-operative pain has been identified as the only factor that significantly predicts post-thoracotomy pain syndrome (Katz, J. et al Clinical J. Pain (1996); 12:50-5). Administration of opioids and local anesthetic agents singly or in combination has been the mainstay of therapy, but to date no optimal analgesic strategy has emerged.

New and improved compounds and methods of treating pain are an on-going requirement in human medicine and, indeed, in the treatment of pain in other vertebrates and is the subject of extensive research.

### SUMMARY OF INVENTION

The invention provides a method of reducing the perception of pain in a subject, which comprises administering to said subject an effective amount of a substance which is COX-2 selective and which can reduce prostaglandin levels of cerebrospinal fluid (CSF).

Thus, the invention provides a means of controlling pain in those in need thereof.

Preferably, the amount of a narcotic administered separately, sequentially or simultaneously can be reduced relative to the amount normally administered so as to achieve satisfactory analgesia.

A preferred COX-2 selective inhibitor is nimesulide.

The narcotic is preferably an opioid, for example, morphine.

The invention has particular application in the relief of pain following surgery, especially following thoracotomy.

The invention is also useful in the treatment of chronic post-operative pain, especially chronic post thoracotomy pain as hereinafter described.

An increase in prostaglandin generation in response to nociceptive signals in the spine may be due to an induction of cyclooxygenase (COX)-2 in the dorsal ganglia. Consequently, selective COX-2 inhibitors may be particularly useful for pain relief following surgery, especially as they have been found to have a reduced potential for injuring gastric mucosa and inhibiting haemostasis.

Post-thoracotomy pain and increase in CSF prostaglandin levels were found herein to be suppressed by a selective COX-2 inhibitor. Thus, much of the pain perceived following thoracotomy and poorly suppressed by narcotic analgesics may be mediated by COX-2 dependent prostaglandin generation in the neurones of the spinal cord.

### BRIEF DESCRIPTION OF DRAWINGS

FIG. 1 is a graph of serum thromboxane B₂ (TXB₂ (ng/ml)) versus time (days) following the administration of ibuprofen (- [symbol] -) and nimesulide (-▪-) relative to a control (-⋄-);

FIG. 2 is a graph of plasma PGE₂ (ng/ml) versus time (days) following the administration of ibuprofen ( [symbol] ) and nimesulide (-▪-) relative to a control (-⋄-);

FIG. 3 is a series of box plots of CSF 6-keto-PGF_{1α} (pg/ml) versus time (days) following administration of ibuprofen and nimesulide relative to a control as described in the Example;

FIG. 4 is a series of box plots showing pain scores at rest and on coughing on a visual analog score (VAS) following administration of nimesulide and ibuprofen and in a control as described in the Example;

FIG. 5 is a series of box plots depicting the requirement for intrathecal morphine requirement as described in the Example; and

FIG. 6 is a series of box plots depicting peak expiratory flow rate (PEFR) on day 2 as a percentage of the preoperative value as described in the Example.

The invention will be further illustrated with reference to the accompanying Example.

### MODE FOR CARRYING OUT THE INVENTION

### EXAMPLE

To determine whether COX-2 plays a role in nociception in humans, we examined the effects of a selective COX-2 inhibitor nimesulide on pain control and CSF prostaglandin concentrations in patients undergoing thoracotomy.

Thirty patients undergoing thoracotomy for adenocarcinoma were randomized to three groups receiving: i) no NSAIDs; ii) the non-selective COX inhibitor ibuprofen; or iii) the COX-2 selective inhibitor nimesulide as part of their analgesic regime in conjunction with intrathecal morphine. CSF was analysed for 6-keto-prostaglandin (PG)F_{1α}, the principal metabolite of prostacyclin. COX-1 and COX-2 activity were determined ex vivo by measuring serum thromboxane (TX)B₂ and endotoxin-induced PGE₂ generation, respectively in whole blood. Pain was assessed using a visual analogue scale (VAS), intrathecal morphine requirement and peak expiratory flow rate as described below in greater detail.

Subjects

The study was approved by the Irish Medicines Board and by the Ethics Committee of St. James Hospital, Dublin and all patients gave informed, written consent. The thirty patients recruited had had an elective lobectomy or pneumonectomy for bronchial carcinoma. Exclusion criteria included a history of peptic ulcer disease, renal and hepatic dysfunction, psychiatric illness, any chronic pain syndrome and consumption of NSAIDs, corticosteroids or any other drug known to interfere with PG production for 14 days prior to surgery.

As indicated above, the thirty patients, 17 males and 13 females with a mean age of 63 years, were randomly assigned in an open label manner to one of three groups. The following table shows the demographic data.

Demographic data and operative procedures

| Characteristics | Control | Ibuprofen | Nimesulide |
|---|---|---|---|
| Patient Numbers | 10 | 10 | 10 |
| Male | 6 | 5 | 6 |
| Mean Age (yrs) | 62.8 | 63.3 | 64.1 |
| Mean Weight (kg) | 70.6 | 72.6 | 64.0 |
| Pneumonectomy | 2 | 2 | 4 |
| Lobectomy | 8 | 8 | 6 |

As indicated above, Group i) (control) received no NSAIDs; Group ii) received the COX-2 selective inhibitor nimesulide 100 mg twice daily; and Group iii) received the non-selective COX inhibitor, ibuprofen 400 mg four times daily. The first dose of the COX inhibitor was administered just prior to induction of general anesthesia.

An intrathecal catheter (22G spinal catheter, B. Braun Medical Melsungenag D/34209) was placed through the L₃₋₄ interspace. Two ml of CSF was withdrawn preoperatively and then once every 24 h. postoperatively, the first 500 μL of CSF being discarded as the catheter dead-space was 300 μL. At the same times, 6 ml of peripheral venous blood was obtained. All patients received morphine 1 mg via the catheter prior to incision. This catheter was used for delivery of morphine for postoperative analgesia and was removed at 48 h. once the final CSF sample had been obtained.

Standardised anaesthetic and surgical techniques were used. The incision was made above or below the sixth rib. No NSAIDs were administered intraoperatively and the only opioid used was fentanyl 100-200 μg as an intermittent bolus at the discretion of the anesthesiologist.

Biochemical Analysis

Mass spectrometric analysis showed that the major product in human CSF was 6-keto-PGF_{1α}, the immediate hydrolysis product of prostacyclin, with only small amounts of PGE₂ detected (data not shown). Thereafter, the CSF was analysed for 6-keto-PGF_{1α} by enzyme immunoassay (EIA) (R&D Systems, Minneapolis, Minn.).

COX-1 and COX-2 activities were analysed in whole blood. Briefly, non-anticoagulated whole blood was allowed to clot in a non-siliconised glass tube at 37° C. for 1 h. Serum was separated by centrifugation at 1000× g for 10 mins and stored at −20° C. Serum TXB₂ was determined as an assay of COX-1 activity by EIA. In addition, a 1 ml aliquot of whole blood containing 10 IU of sodium heparin was incubated in the presence of lipopolysaccharide (LPS) derived from Escherichia Coli (Sigma Chemical Co.) 10 μg/ml at 37° C. for 24 h. The contribution of platelet COX-1 was suppressed by the addition of 200 μM aspirin. Plasma was separated by centrifugation at 1000× g for 10 min. and stored at −20° C. PGE₂ was measured by EIA as an index of COX-2 activity according to the method of Patrignani, P., et al (Journal of Pharmacology and Experimental Therapeutics (1994); 271:1705-1712).

Patient Monitoring

After surgery the patients were returned to the High Dependancy Unit, extubated and with intrathecal catheter in situ. Pain score assessments were made following administration of analgesic (VAS<3) with the COX inhibitor and intrathecal morphine 0.5-1 mg prn. The amount of morphine required throughout the day was recorded and pain was assessed using a 10 point visual analogue score at rest (VAS-R) and upon coughing (VAS-C). On the morning of the second postoperative day, 2 hours after the administration of the drug being studied the average of three peak flow readings was obtained using a portable hand held peak flow meter and compared to preoperative values.

Statistical Analysis

The BOX-COX transformation diagnostic procedure was applied to all measured response variables. Where multiple responses were measured on study participants a repeated measure analysis of variance and associated Student t tests of linear contrasts among the means were used. In the case where a single response was measured a one way analysis of variance and associated Students t tests were used. The p values quoted in the text are all single tail values.

Results

Prostaglandin Formation

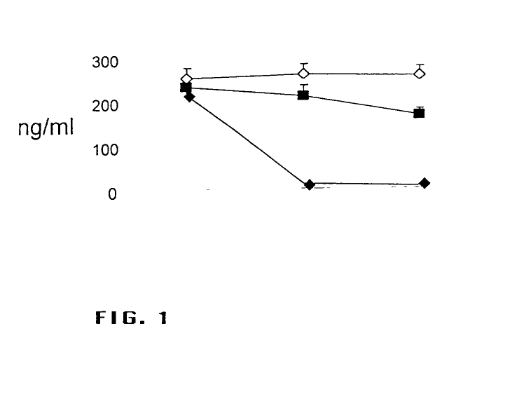

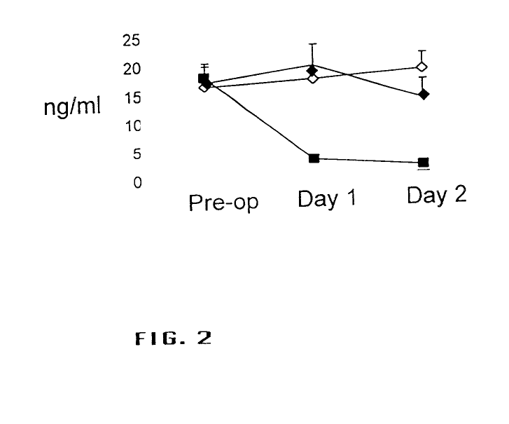

Serum TXB₂, a marker of COX-1 activity was markedly suppressed by ibuprofen (4.5±1.1 ng/ml on day 2 vs 180±24 ng/ml before treatment), whereas nimesulide had little effect (171±20 ng/ml on day 2 vs 220±19 ng/ml before treatment). LPS-induced PGE₂ formation in whole blood, an index of COX-2 activity, was markedly inhibited by nimesulide (1.2±0.2 ng/ml on day 2 vs 17.3±2.5 ng/ml before treatment), whereas ibuprofen had little effect (12.9±3.3 ng/ml on day 2 vs 16.4±2.8 ng/ml before treatment). Serum TXB₂ and LPS-induced plasma PGE₂ were unaltered in the control group of patients that did not receive a cyclooxygenase inhibitor as shown in FIGS. 1 and 2.

FIG. 1 depicts the effect of ibuprofen and nimesulide on serum TXB₂ (COX-1 activity) compared with no treatment (control). The effect of ibuprofen and nimesulide on endotoxin-induced plasma PGE₂ (COX-2 activity) compared with no treatment (control) is depicted in FIG. 2. In each of FIGS. 1 and 2 the data are presented as mean ±SEM for the preoperative period and for days 1 and 2. It will be observed from FIGS. 1 and 2 that ibuprofen was largely a COX-1 inhibitor, whereas nimesulide was selective for COX-2.

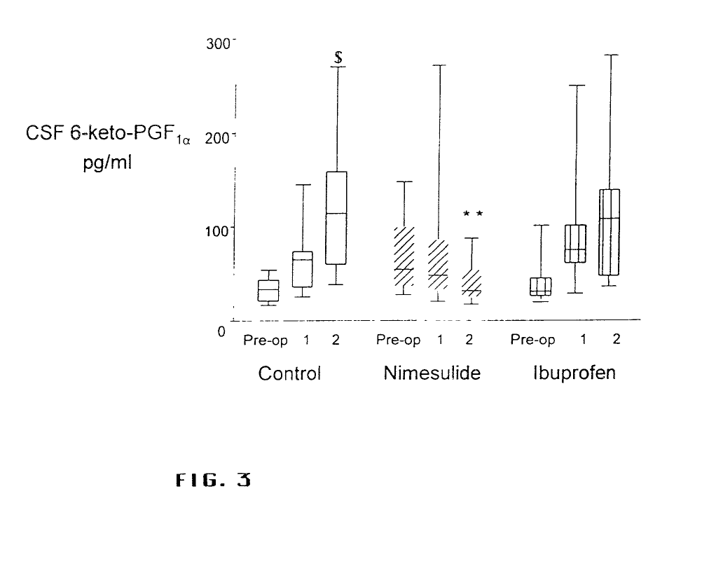

FIG. 3 shows the concentration of 6-keto-PGF_{1α} in the CSF at the time of and on the two days following surgery. The data are presented as box plots showing the median and the 75% and 95% confidence intervals. There was a marked rise in untreated, control patients following surgery (from 32±4.9 to 127±29.3 pg/ml; p<0.001) that was blunted in patients treated with nimesulide (49±9.3 pg/ml on day 2; p=0.0025 vs control). In contrast, ibuprofen had no effect (122±35.2 pg/ml on day 2).

Analysis of Quality of Analgesia

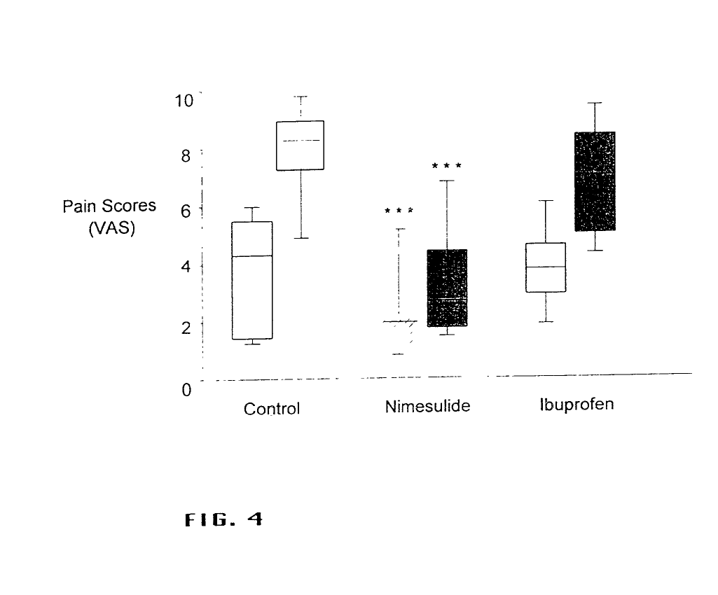

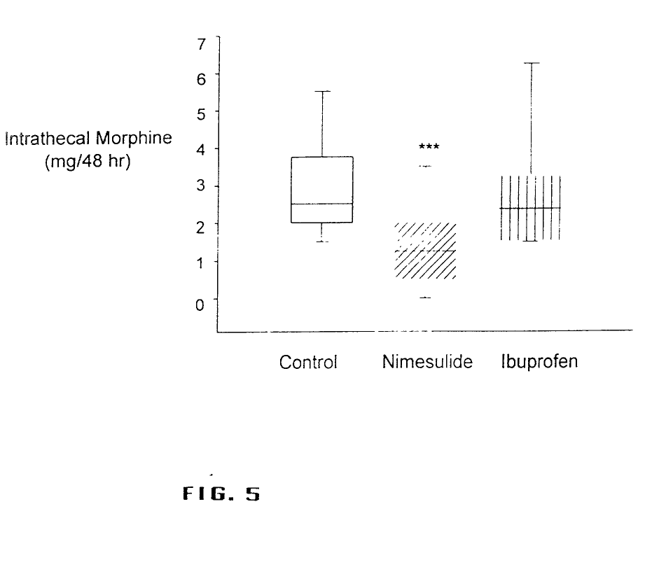

FIG. 4 depicts assessment of pain relief bases on a visual analogue score on day 1 and day 2 following surgery. The data are presented as box plots showing the median and the 75% and 95% confidence intervals. Pain scores at rest and immediately after coughing were lower in the nimesulide group over the forty eight hours following surgery. The intrathecal morphine requirements were also lower in the nimesulide group compared with the control group (p=0.0175), whereas again ibuprofen had no effect as shown in FIG. 5. Again the data are presented box plots showing the median and the 75% and 95% confidence intervals.

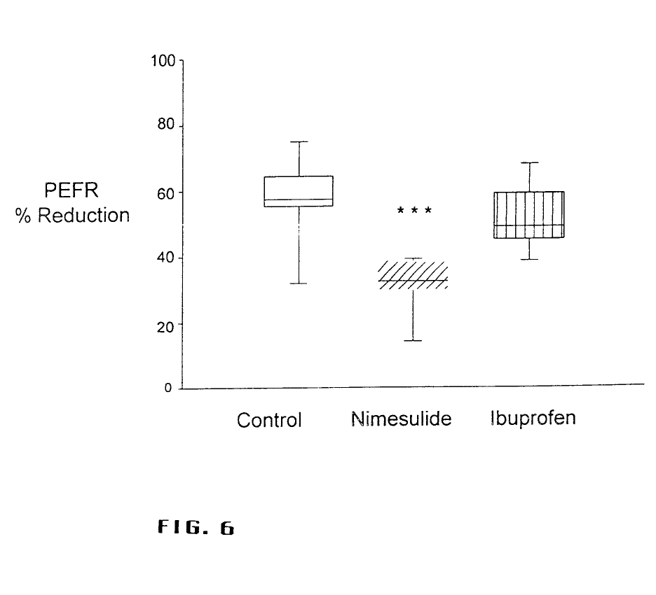

FIG. 6. depicts peak expiratory flow rate (PEFR) on day 2 expressed as a percent of the preoperative value. The data are presented as box plots showing the median and the 75% and 95% confidence intervals. The reduction in peak expiratory flow rate was blunted by nimesulide (p<0.001 vs control) but not by ibuprofen.

The above results suggest that COX-2 is primarily responsible for spinal prostaglandin formation following thoracotomy. Thoracotomy was associated with a rise in CSF 6-keto-PGF_{1α} in the 48 h. following surgery. The increase is likely to represent local generation as the levels far exceed those seen in plasma. The increase was COX-2 dependent as it was suppressed by nimesulide. The selectivity of nimesulide as a COX-2 inhibitor (Patrignani, P., et al supra) was confirmed in this study. In contrast, at the dose used ibuprofen was largely an inhibitor of COX-1 and had no effect on CSF 6-keto-PGF_{1α}. The results are consistent with in vitro studies demonstrating that ibuprofen has a 2-15 fold selectivity for COX-1 over COX-2 (Mitchell, J. A. et al Proceedings of the National Academy of Science USA (1994); 90:11693-7), although other work suggests that ibuprofen is equally effective against both isoforms (Meade, E. A. et al J. Biol. Chem. (1993); 268:6610-4).

The above results show that COX-2 inhibition and suppression of CSF 6-keto-PGF_{1α} improves the pain relief achieved over that seen with opioid analgesic alone. The improved analgesia was associated with a reduced requirement for intrathecal morphine, and probably explains the improved respiratory function following surgery. The results suggest that suppression of COX-2 is responsible for the observed enhancement of opioid analgesia by NSAIDs.

Thus, the above results provide evidence of a role for cyclooxygenase in spinal nociceptive processing. In particular, the results suggest that COX-2 is the major isoform involved. As COX-2 inhibitors are associated with less gastric injury (at least acutely) and do not influence haemostasis (Hawkey, C. J. Lancet (1999); 23;353:307-14), they may represent a safer alternative to NSAIDs in the management of postoperative pain.

1. A method of reducing the perception of pain in a subject, which comprises administering to said subject an effective amount of a substance which is COX-2 selective and which can reduce prostaglandin levels of cerebrospinal fluid.

2. A method according to claim 1, wherein the amount of a narcotic drug administered separately, sequentially or simultaneously can be reduced relative to the amount normally administered so as to achieve satisfactory analgesia.

3. A method according to claim 1 or 2, wherein the substance is nimesulide.

4. A method according to claim 1 or 2, wherein the narcotic drug is an opioid.

5. A method according to claim 4, wherein the opioid is morphine.

6. A method according to claim 1 or 2, for the relief of pain following surgery.

7. A method according to claim 6, for the relief of pain following thoracotomy.

8. A method according to any claim 1 or 2, for the treatment of chronic post-operative pain.

9. A method according to claim 8, for the treatment of chronic post-thoracotomy pain.
